(19) United States
(12) Patent Application Publication
(10) Pub. No.: US 2026/0215836 A1
(43) Pub. Date: Jul. 30, 2026

(54) DEBRIS TRAP FOR ELECTROSURGICAL PENCILS WITH SMOKE EVACUATION SYSTEM

(71) Applicant: Dayton Medical Innovations, LLC, Oakwood, OH (US)

(72) Inventor: William Carrigg

(21) Appl. No.: 19/398,977

(22) Filed: Nov. 24, 2025

Related U.S. Application Data
(60) Provisional application No. 63/733,553, filed on Dec. 13, 2024.

(51) Int. Cl. A61B 18/14; A61B 18/00

(52) U.S. Cl. CPC A61B 18/14; A61B 2018/00172; A61B 2018/00184; A61B 2018/00601; A61B 2018/1412; A61B 2018/1475; A61B 2218/008

(57) ABSTRACT

Embodiments provided herein include a smoke evacuation electrosurgical pencil. Some embodiments include a vacuum tube that is substantially cylindrical and includes an inner surface. The vacuum tube may be configured for vacuuming debris during use of the smoke evacuation electrosurgical pencil in a surgical procedure. Also included is an electrosurgical electrode blade that is partially disposed within the vacuum tube and partially extending from an end of the vacuum tube. The electrosurgical electrode blade may be configured for performing the surgical procedure and may include a shaft that is disposed in the vacuum tube. Some embodiments include a debris trap coupled to the shaft and removably disposed within the vacuum tube. The debris trap may extend to the inner surface of the vacuum tube and include a plurality of trapping structures to receive and capture debris resulting from the surgical procedure.

### CROSS-REFERENCE TO RELATED APPLICATIONS

The present disclosure claims the benefit of U.S. Provisional Patent Application No. 63/733,553, filed December 13, 2024, entitled "Debris Trap for Electrode in Electrosurgical Pencils with Smoke Evacuation System", and U.S. Provisional Patent Application No. 63/778,740, filed March 27, 2025, entitled "Debris Trap for Electrosurgical Pencils with Smoke Evacuation System" the entirety of each being incorporated by reference herein.

### TECHNICAL FIELD

The present application generally relates to electrosurgical pencils with a smoke evacuation system and, more specifically, debris traps for electrosurgical pencils with a smoke evacuation system.

### BACKGROUND

Electrosurgical instruments are integral to a wide range of surgical procedures, performing functions such as tissue dissection, debridement (removal of damaged tissue), and vessel cauterization (sealing blood vessels by burning). These instruments typically comprise a pencil- shaped handle and in modern designs, specialized electrode blades. The electrode blades deliver high- frequency alternating current to the tissue, enabling both cutting and cauterization. As a result, this process generates a significant amount of surgical smoke, a hazardous byproduct that contains toxic chemical compounds, viruses, ultra-fine particles, and even living cells. Surgical smoke is not only released directly at the surgical site but also disperses into the surrounding air of the operating room and can travel through the hospital's ventilation system, posing a potential risk to healthcare workers and patients.

Recognizing the dangers of surgical smoke, 18 U.S. states, and several other regions, have passed laws mandating the use of surgical smoke evacuation systems during electrosurgical procedures. These evacuation systems are typically built into electrosurgical pencils and work by positioning a vacuum mechanism near the surgical blade. This setup allows the smoke to be immediately evacuated from the surgical site, reducing the risk of exposure and contamination. The systems rely on high-efficiency vacuum suction combined with catalytic filters, which help capture and neutralize the harmful components of the surgical smoke before it can circulate in the operating room environment. While effective at reducing airborne contaminants, these systems face several challenges related to the evacuation of surgical smoke.

One of the major challenges is that the high vacuum pressure required to evacuate surgical smoke can also cause larger debris, such as cauterized tissue, clotted blood, and loose tissue, to be sucked into the electrosurgical pencil assembly. This debris accumulation causes blockages within the pencil's internal components, impairing the functionality of the surgical smoke evacuation system. As the blockage builds up, the suction power reduces, leading to longer surgical times, the need for frequent interruptions to clear the system, and an increased risk of infection due to the potential contamination in the operating room.

Efforts to manage and resolve these blockages are needed to maintain a smooth and safe surgical process. In many cases, blockages are addressed using manual methods by the surgical team. These might include the use of gauze, surgical brushes, or even surgical assistants who are trained to handle clogging issues swiftly. However, this process is labor-intensive, time-consuming, and can still result in delays, which compromises the overall efficiency and safety of the procedure. Therefore, electrosurgical instrument blockages should be resolved quickly in a way that minimizes disruptions and ensures patient safety.

Some manufacturers have attempted to address blockages by increasing the bore size of the electrosurgical pencil assembly. A larger bore allows larger debris to pass through and facilitates higher vacuum flow, which can evacuate the surgical smoke more effectively. However, while these larger instruments solve the problem of suction power, they introduce new challenges. The increased bore size requires more powerful vacuum systems, larger tubing, and bulkier pencil assemblies, making them unwieldy and difficult to use in many modern operating rooms. These larger instruments also limit the versatility of electrosurgical tools, restricting their use in smaller or more intricate surgeries, where precision and maneuverability are critical.

Despite these attempts, the most common scenario in modern operating rooms involves surgeons and their teams relying on their ingenuity to prevent blockages, clear clogs, and keep the procedure moving forward. This typically involves a combination of manual tools, such as gauze or surgical brushes, and the quick thinking of the surgical staff. However, this manual approach is far from ideal, and there remains a significant need for a more effective, streamlined, and automated solution to address the issue of blockages without disrupting the surgical flow.

### SUMMARY

Embodiments provided herein include a smoke evacuation electrosurgical pencil. Some embodiments include a vacuum tube that is substantially cylindrical and includes an inner surface. The vacuum tube may be configured for vacuuming debris during use of the smoke evacuation electrosurgical pencil in a surgical procedure. Also included is an electrosurgical electrode blade that is partially disposed within the vacuum tube and partially extending from an end of the vacuum tube. The electrosurgical electrode blade may be configured for performing the surgical procedure and may include a shaft that is disposed in the vacuum tube. Some embodiments include a debris trap coupled to the shaft and removably disposed within the vacuum tube. The debris trap may extend to the inner surface of the vacuum tube and include a plurality of trapping structures to receive and capture debris resulting from the surgical procedure.

One embodiment of a smoke evacuation electrosurgical pencil may include a vacuum tube that is substantially cylindrical and includes an inner surface. The vacuum tube may be configured for vacuuming debris during use of the smoke evacuation electrosurgical pencil in a surgical procedure. Some embodiments include an electrosurgical electrode blade that is partially disposed within the vacuum tube and partially extending from an end of the vacuum tube. The electrosurgical electrode blade may be coupled to the smoke evacuation electrosurgical pencil by a coupling mount that extends from a wall of the vacuum tube. The electrosurgical electrode blade may be configured for performing the surgical procedure and may include a shaft that is disposed in the vacuum tube. Some embodiments include a debris trap coupled to the shaft and removably disposed within the vacuum tube. The debris trap may extend to the inner surface of the vacuum tube and include a plurality of trapping structures to receive and capture debris resulting from the surgical procedure.

Another embodiment of a smoke evacuation electrosurgical pencil may include a vacuum tube that is substantially cylindrical and includes an inner surface. The vacuum tube may be configured for vacuuming debris during use of the smoke evacuation electrosurgical pencil in a surgical procedure. Some embodiments include an electrosurgical electrode blade that is partially disposed within the vacuum tube and partially extends from an end of the vacuum tube. The electrosurgical electrode blade coupled to the smoke evacuation electrosurgical pencil by a coupling mount that extends from a wall of the vacuum tube. The electrosurgical electrode blade may be configured for performing the surgical procedure and may include a shaft that is disposed in the vacuum tube. Some embodiments include a debris trap coupled to the shaft and removably disposed within the vacuum tube, the debris trap extending to the inner surface of the vacuum tube. The debris trap may include a plurality of trapping structures to receive and capture debris resulting from the surgical procedure. The debris trap may include a coupling groove for engaging with a notch on the shaft.

These and additional features provided by the aspects described herein will be more fully understood in view of the following detailed description, in conjunction with the drawings.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1 depicts a standard electrosurgical electrode blade, according to embodiments provided herein;

FIG. 2 depicts a side view of the electrode blade with a ring-like debris trap, according to embodiments provided herein;

FIG. 3 depicts an oblique view of the electrode blade with a ring-like debris trap, according to embodiments provided herein;

FIG. 4 depicts an axial view of the electrode blade with a ring-like debris trap, according to embodiments provided herein;

FIG. 5A depicts an electrode blade with a debris trap that has a fan blade like fin structure, according to embodiments provided herein;

FIG. 5B depicts a debris trap having a fan blade like fin structure 6 with no outer ring, according to embodiments provided herein;

FIG. 6 is an electrode blade with a debris trap that has a variation of its fan blade fin structure, in which the fins have a tapered structure, according to embodiments provided herein;

FIG. 7A depicts a variation of the electrode blade with a debris trap that includes a plastic straw brush-like fin structure that allows surgical smoke to pass but catch debris, according to embodiments provided herein;

FIG. 7B depicts a debris trap including the plastic straw brush-like fin structure, according to embodiments provided herein;

FIG. 8 depicts a variation of the electrode blade with a debris trap that have a rectangular outer ring designed for a rectangular vacuum tube, according to embodiments provided herein;

FIG. 9 depicts an electrode blade with a ring-like debris trap having an outer ring 4 and an inner ring, according to embodiments provided herein;

FIG. 10 depicts the electrode blade with ring-like debris trap while in an electrosurgical pencil with smoke evacuation system, according to embodiments provided herein;

FIG. 11 depicts a standard smoke evacuation electrosurgical pencil, which has become clogged with debris, according to embodiments provided herein;

FIG. 12 depicts the electrode blade with ring-like debris trap that has been removed from the pencil and cleared of debris, according to embodiments provided herein;

FIG. 13 depicts a standard smoke evacuation electrosurgical pencil which is now functional once again after having debris removed. No surgical smoke is contaminating the air, according to embodiments provided herein;

FIG. 14 depicts a smoke evacuation electrosurgical pencil, including an electrode blade with a plate-like debris trap 14 that includes circular holes, according to embodiments provided herein;

FIG. 15 depicts a variation of fin design with forward tapered fins, according to embodiments provided herein;

FIG. 16 depicts an axial view of a variation with forward tapered fins, according to embodiments provided herein;

FIG. 17 depicts a cup/cage construct that acts as a filter, according to embodiments provided herein;

FIG. 18 depicts a non-concentric electrode placement within the trap, according to embodiments provided herein;

FIG. 19 depicts a variation with curved fins, according to embodiments provided herein;

FIGS. 20A and 20B depict a variation with a notched cutout on the fin edge, according to embodiments provided herein;

FIG. 21 depicts a variation with fins and a ring, according to embodiments provided herein;

FIG. 22 depicts a variation with tines that extend from the fin at a substantially perpendicular angle, according to embodiments provided herein;

FIG. 23 depicts a variation with forking at the outer edge of the fin and plurality of prongs, according to embodiments provided herein;

FIG. 24 demonstrates that the entire debris trap structure can be manufactured separately from the insulative cover on the shaft, according to embodiments provided herein;

FIG. 25 depicts a CAD drawing of a debris trap with fins that have chamfered cuts on upper and lower edge of fins, according to embodiments provided herein;

FIG. 26 depicts a CAD drawing of a debris trap with fins that have chamfered cuts on upper and lower edge of fins, according to embodiments provided herein;

FIG. 27 depicts a CAD drawing of a debris trap with fins that do not have chamfered cuts, according to embodiments provided herein;

FIG. 28 depicts a CAD drawing of a debris trap with fins that do not have chamfered cuts, according to embodiments provided herein;

FIG. 29 depicts a side view of a debris trap 200 with fins that do not have chamfered cuts, according to embodiments provided herein;

FIG. 30 depicts a standard electrosurgical pencil with smoke evacuation system, according to embodiments provided herein;

FIG. 31 depicts an electrosurgical electrode with the debris trap at an asymmetric point between the male connection end 1 and the blade portion, according to embodiments provided herein;

FIG. 32 depicts an electrosurgical electrode with the debris trap at an asymmetric point between the male connection end 1 and the blade portion, according to embodiments provided herein;

FIG. 33A depicts a side view of a curved net that is connected to an attachment component, according to embodiments provided herein;

FIG. 33B depicts another side view of the curved net-like debris trap of FIG. 33A, according to embodiments provided herein;

FIG. 34A depicts an example locking mechanism for securing a debris trap that is configured to removably couple to the electrode, according to embodiments provided herein;

FIG. 34B depicts a separate debris trap with a bayonet-style coupling groove into which a peg from FIG. 34A can securely engage and lock, according to embodiments provided herein;

FIG. 34C depicts the separate debris trap fully coupled and locked onto the peg, according to embodiments provided herein;

FIG. 35 depicts another embodiment of a debris trap, according to embodiments provided herein;

FIG. 36 depicts another embodiment of a debris trap with prongs and a securing portion that engages with the shaft, according to embodiments provided herein;

FIG. 37A and 37B depict an electrode debris trap that includes fins and vanes existing between the fins, according to embodiments provided herein;

FIGS. 38A and 38B depict a debris trap comprising multiple rows of fins, according to embodiments provided herein;

.FIGS. 39A and 39B depict a smoke evacuation electrosurgical pencil with a cutout window outlined by dotted lines, illustrating the vacuum tube interior, according to embodiments provided herein;

FIGS. 40A, 40B, 40C, and 40D depict multiple views of an insulative electrode shaft cover comprising a debris trap without an electrode present, according to embodiments provided herein;

FIG. 41A depicts an axial view of a removable vacuum tube incorporating a debris trap without an electrode, according to embodiments provided herein;

FIG. 41B depicts an oblique view of the debris trap of FIG. 41A without an electrode. The internal lumen is dimensioned to accommodate an electrosurgical electrode blade, according to embodiments provided herein;

FIG. 41C depicts a smoke evacuation pencil fitted with a removable vacuum tube containing a debris trap, depicted without an electrode, according to embodiments provided herein;

FIG. 42A depicts a smoke evacuation electrosurgical pencil featuring a built-in debris trap positioned around the vacuum tube lumen opening, according to embodiments provided herein;

FIG. 42B depicts the vacuum cap detached from the smoke evacuation electrosurgical pencil, according to embodiments provided herein;

FIG. 42C depicts an axial view of the electrosurgical pencil with built in debris trap highlighting the vacuum tube lumen as indicated by the shaded area, according to embodiments provided herein;

FIGS. 43A through 43D depict an embodiment of an electrosurgical pencil incorporating a smoke evacuation system with an integrated electrode connector sheath, according to embodiments provided herein;

FIG. 43B depicts a similar embodiment as provided in FIG. 43A, with the electrode removed to better show the internal structure of the sheath and lumen, according to embodiments provided herein;

FIG. 44A depicts an electrosurgical pencil featuring a non-concentric vacuum tube lumen, according to embodiments provided herein; and

FIG. 45 depicts an electrosurgical pencil featuring a net-like debris trap positioned over the vacuum tube lumen and coupled to an electrode, according to embodiments provided herein.

### DETAILED DESCRIPTION

Embodiments provided herein include a debris trap for electrosurgical pencils with a smoke evacuation system for use in a surgical procedure. These embodiments incorporate the debris trap directly into the central shaft of the electrode blade. The debris trap is designed to capture and contain larger debris before the debris enters the electrosurgical pencil assembly, preventing blockages that could otherwise disrupt the surgical smoke evacuation system. The design of the electrode blade allows the electrode blade to be easily removed, replaced, and interchanged for different surgical procedures, providing the surgical team with flexibility and adaptability in their toolset.

For example, by incorporating the debris trap flush on outer shaft of the electrode blade and connecting it to the surgical smoke evacuation tube within the electrosurgical pencil, the system enables a quick and simple push-pull method for clearing debris. Surgeons can easily remove or replace the blade without interrupting the flow of the procedure. This innovative feature not only improves the performance of the surgical smoke evacuation system but also significantly reduces the likelihood of blockages and minimizes the need for time-consuming manual interventions.

The debris trap structure may be manufactured separately and configured to slide on and off the electrode blade or any conventional or yet-to-be developed electrodes recognized by those skilled in the art.

With this approach, surgical teams can focus more on the patient and less on maintaining the functionality of the electrosurgical instruments without being exposed to toxic surgical smoke. The ability to quickly clear blockages without requiring extensive downtime leads to a more efficient surgical process, ultimately improving patient outcomes, reducing surgical time, and minimizing the risk of infection. Furthermore, the design remains adaptable to a wide variety of surgical contexts, offering a more agile and user-friendly solution to a common problem faced in operating rooms around the world. This invention represents a major step forward in enhancing the functionality and safety of electrosurgical tools, providing a cleaner, more effective environment for both patients and healthcare professionals.

As used herein, the word "example" means an instance, or illustration. The word "example" does not indicate a key or preferred aspect or aspect. The word "or" is intended to be inclusive rather than exclusive unless context suggests otherwise. As an example, the phrase "A employs B or C," includes any inclusive permutation (e.g., A employs B; A employs C; or A employs both B and C). As another matter, the articles "a" and "an" are generally intended to mean "one or more" unless context suggest otherwise.

It is noted that the terms "substantially," "about," and "approximately" may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

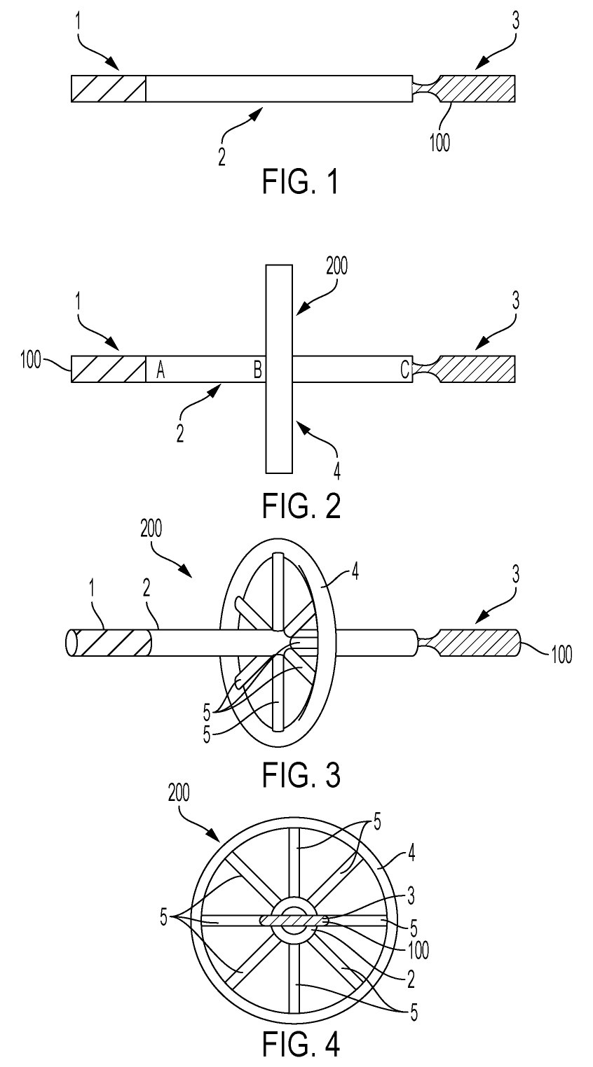

FIG. 1 depicts an electrosurgical electrode blade 100. The electrosurgical electrode blade 100 includes a male connection end 1 for connection to an electrosurgical pencil. A shaft 2 of conductive material on the electrosurgical electrode blade 100 is covered by an insulative cover that resists electric and thermal energy as well as abrasion. The insulative cover may be shrink wrapped or otherwise adhered to the shaft 2. A blade portion 3 of the electrosurgical electrode blade 100, opposite the male connection end 1, may cut or coagulate tissue depending on the type of electric current.

FIG. 2 depicts a side view of the electrosurgical electrode blade 100 with a debris trap 200 in a ring-like configuration. The outer ring 4 is circumferential to the electrosurgical electrode blade 100. The debris trap 200 may include an insulative polymer including, but not limited to, PS, PVC, PEEK, PTFE, PEI, PPSU, PPS, LCP, PAI, PC, Nylon, PP, or ABS. The electrode blade may have a coating of a variety of different materials including but not limited to PTFE, silicon nitride, gold, silver, nitinol, silicone ceramic, stainless steel, or an elastomeric coating. The electrosurgical electrode blade 100 may be configured as an electrical conductor, which may be made of any of a variety of materials which includes but is not limited to stainless steel, nickel, platinum, copper, zinc, gold, silver, or any other metal. The debris trap 200 may be formed integrally with the insulative cover, or it may be a separate component attached to the cover using over-molding, press fitting, adhesive bonding, and/or other suitable joining techniques."

FIG. 3 depicts an oblique view of the electrosurgical electrode blade 100 with a debris trap 200 in a ring-like configuration. The fins 5 connect the outer ring 4 of the debris trap 200 to the shaft 2. The fins 5 may be spaced out to allow surgical smoke to pass through but catch larger debris. There may be any number of fins and multiple outer rings.

FIG. 4 depicts an axial view of the electrosurgical electrode blade 100 with a debris trap 200 in a ring-like configuration. As illustrated, the fins 5 may be six in number. Depending on the particular embodiment, more or fewer fins 5 may be utilized.

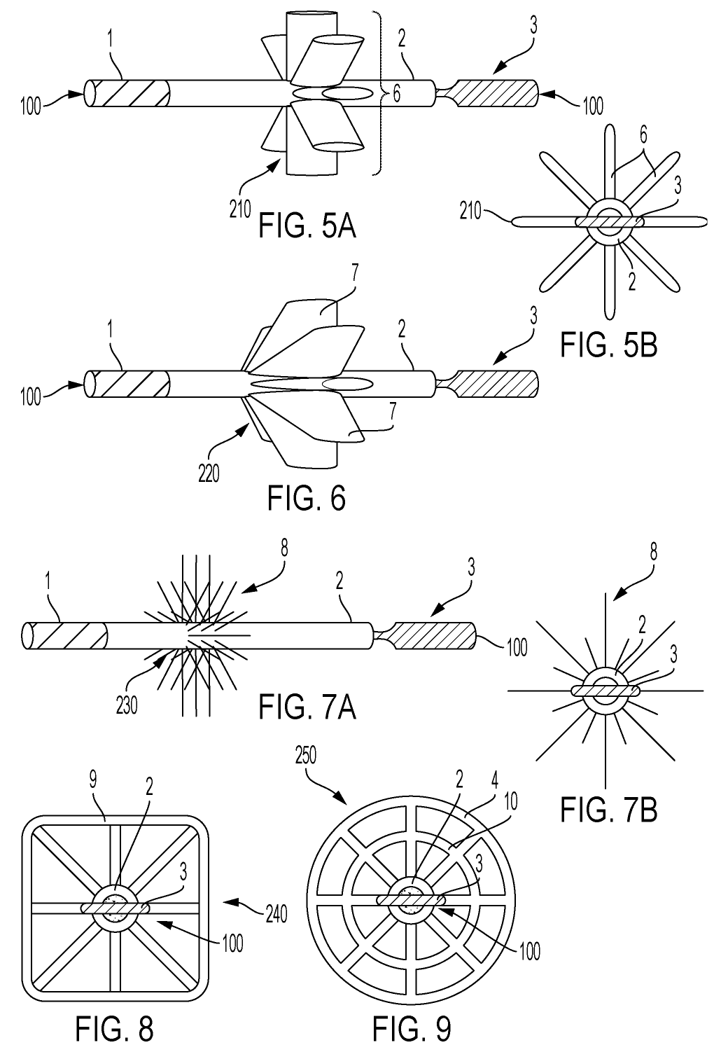

FIG. 5A depicts an electrosurgical electrode blade 100 with a debris trap 210 that has a fan blade like fin structure 6. The edges of the fins may be designed with an aerodynamic geometry. As illustrated, the depicted embodiment does not include an outer ring. FIG. 5B depicts a debris trap 210 having a fan blade like fin structure 6 with no outer ring. Additional embodiments may include other aerodynamic designs including but not limited to at least one curved fin, at least one tapered fin, variable length fins alternating short and long fins, perforated fins, flexible or elastomeric fins, and collapsible fins. Another embodiment could include a radial layout with fins distributed evenly around a central axis, in a spiral or corkscrew layout, angled layout with fins canted in a direction.

FIG. 6 depicts an electrosurgical electrode blade 100 with a debris trap 220 that has a variation of its fan blade fin structure, in which the fins 7 have a tapered structure. This allows for easy replacement of the electrode. Additional embodiments may include rounded or flared tips to prevent snagging. The debris trap 220 may include or coated with a non-stick material and/or a hydrophobic material such as PTFE to reduce adherence of debris and facilitate easier cleaning.

FIG. 7A depicts a variation of the electrosurgical electrode blade 100 with a debris trap 230 that includes a fin structure 8 that may be configured in a plastic straw brush-like configuration and allows surgical smoke to pass but catches debris. FIG. 7B depicts the debris trap 230 including the fin structure 8 from a different perspective. The fin structure 8 may be formed from semi-rigid polymer bristles with variable or consistent bristle densities and thicknesses. Some embodiments may have a plurality of concentric rows of brush like fins.

FIG. 8 depicts a variation of the electrode blade with a debris trap 240 that has a rectangular outer ring 9 designed for a rectangular vacuum tube. Additional embodiments may have geometry which includes but is not limited to triangular, trapezoidal, starburst, or oval.

FIG. 9 depicts an electrode blade with a debris trap 250 with a ring-like configuration having an outer ring 4 and an inner ring 10. Depending on the particular embodiment, the debris trap 250 may include a plurality of inner rings or fan blades. The rings may be stacked axially along the length of the electrosurgical electrode blade 100. The rings may include ventilation slots or perforations to increase airflow. The rings or fins may have surface texturing, grooves, and/or microbarbs to help trap debris from the surgical site.

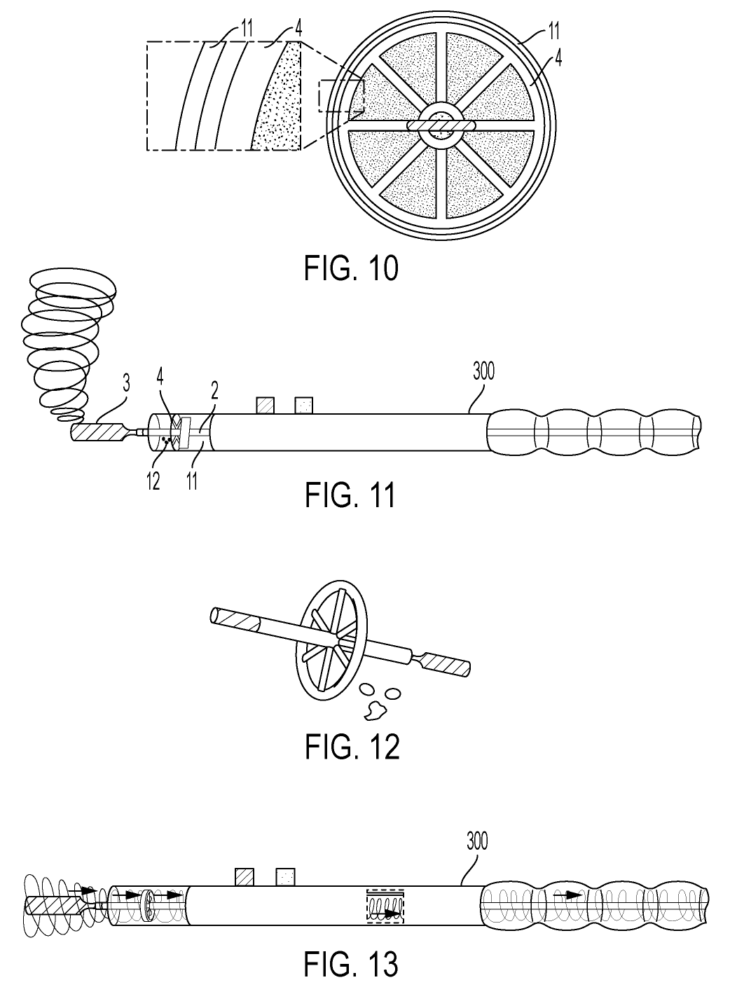

FIG. 10 depicts the blade portion 3 with a debris trap 200 with a ring-like configuration while in an electrosurgical pencil with smoke evacuation system. As illustrated, the outer ring 4 closely matches the inner diameter of the wall of the vacuum tube 11. Blown up section illustrates additional details of outer ring 4 and the wall of the vacuum tube 11.

FIG. 11 depicts a smoke evacuation electrosurgical pencil 300 which has become clogged with debris 12. The debris is lodged in the blade portion 3 with a debris trap 200 in a ring- like configuration. Surgical smoke is being produced by the electrosurgical pencil and is not being suctioned. Toxic surgical smoke is polluting the operating room air. Also illustrated are the blade portion 3, the outer ring 4, the vacuum tube 11, and the shaft 2.

FIG. 12 depicts the blade portion 3 with debris trap in a ring-like configuration that has been removed from the pencil and cleared of debris. As will be understood removing the electrode blade may be performed after a procedure has been completed.

FIG. 13 depicts a smoke evacuation electrosurgical pencil 300 which is now functional once again after having debris removed. No surgical smoke is contaminating the air. Instead, the smoke is drawn into the smoke evacuation electrosurgical pencil 300 in a helical configuration and travels through the smoke evacuation electrosurgical pencil 300.

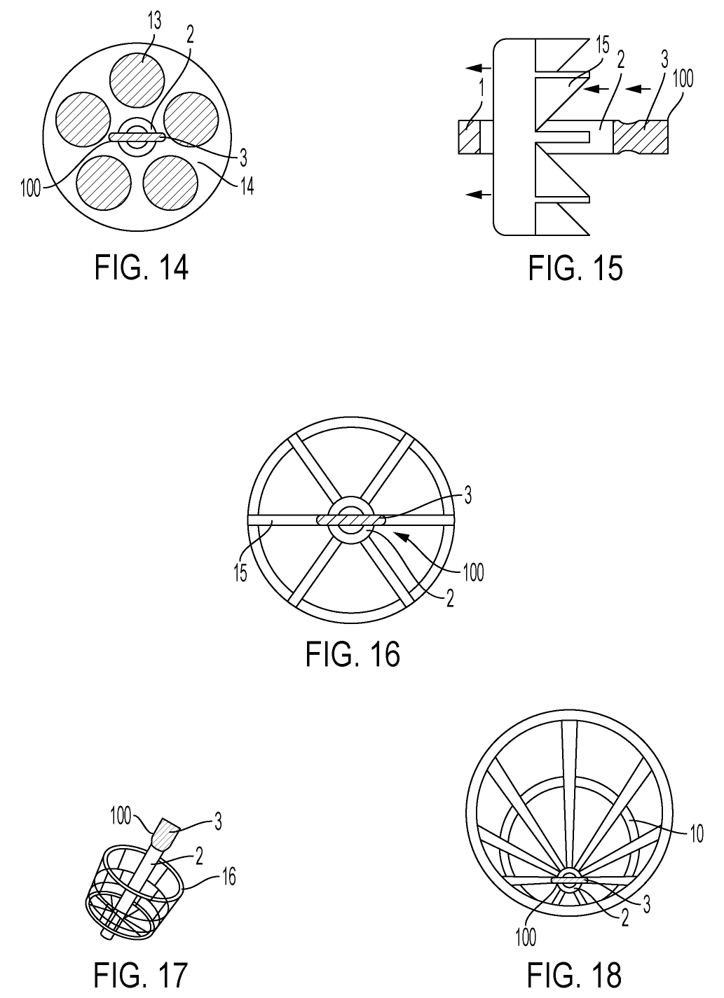

FIG. 14 depicts an electrode blade with a plate-like debris trap 14 that includes circular holes 13 in a disk-like configuration. Additional embodiments may include holes with a triangular, slot-like, or rectangular geometry. Depending on the type of material debris, different shaped holes may be more apt to catch different materials. As an example, if the debris is typically long and slender, the embodiment of FIG. 14 may be preferred, while debris that is round and thick may be better suited with a different shape.

FIG. 15 depicts a variation of fin design with forward tapered fins 15. Some embodiments may include various tapers. As illustrated, the forward tapered fins 15 may be configured to rotate around a center of the electrosurgical electrode blade 100. As such, some embodiments may be configured such that when debris contacts the forward tapered fins 15, the debris trap 200 spins, thereby allowing for an even distribution of debris, which allows for more debris receiving capability.

FIG. 16 depicts an axial view of a variation with forward tapered fins 15. While four forward tapered fins 15 are illustrated, some embodiments may have fewer or more forward tapered fins 15.

FIG. 17 depicts a cup-like configuration 16 that acts as a filter. As illustrated, the cup- like construct 16 may be substantially cylindrical in shape. In some embodiments, the cup-like configuration 16 may have a tapered profile, while some embodiments are not configured in this manner.

FIG. 18 depicts a non-concentric electrode placement within the trap. The electrosurgical electrode blade 100 may be placed at any location within, adjacent to, or outside of the debris trap 200. Such a configuration may be beneficial in scenarios where the position of the electrode dictates that debris will be caught at a predetermined position and a larger area for receiving the debris is desired.

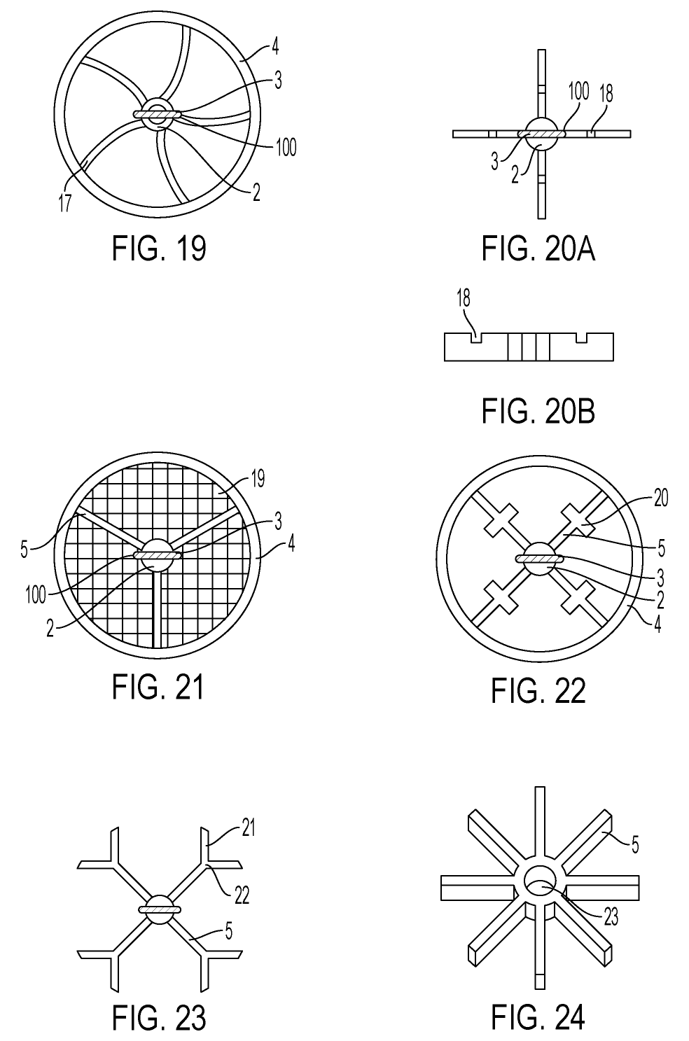

FIG. 19 depicts a variation with curved fins 17. The curved fins 17 may be tapered with wider outer edges. The curvature may take many different forms than depicted in FIG. 19. Additionally, while five curved fins 17 are depicted, more or fewer may be utilized in practice. In some embodiments, not all the curved fins 17 are the same shape. In still some embodiments, curved fins 17 may be utilized with fins of different shapes.

FIGS. 20A and 20B depict a variation with a notched cutout 18 on the fin edge. Depending on the embodiment, the debris trap 200 may include at least one notched cutout for engaging with a notch on the shaft 2. The fin edges may be serrated. As described above for different embodiments, the number of fins may vary, depending on the particular configuration. Additionally, while the embodiment of FIG. 20 illustrates a cross shape, other configurations may be utilized.

FIG. 21 depicts a variation with fins 5 and an outer ring 4. Also depicted is a mesh screen 19 which allows air to pass through but traps debris. As illustrated, the mesh may be configured, based on the expected size of debris that will be collected, balancing airflow with debris collection. Additionally, depending on the embodiment, more or fewer fins 5 may be utilized in varying configurations.

FIG. 22 depicts a variation with tines 20 that extend from the fins 5 at a substantially perpendicular angle. Additional embodiments may include multiple or circular tines. As illustrated, the tines may be utilized for catching debris, while still maintaining airflow. Some embodiments may be configured with connected dines that create a rectangular inner ring or connect only some of the tines 20 together.

FIG. 23 depicts a variation with forking 22 at the outer edge of the fins 5 and plurality of prongs 21. Depending on the embodiment, the debris trap 200 may include at least one prong. Additional embodiments may include multiple areas of forking along the fins 5, curved forks, or serrated fork ends. Again, some embodiments may include mor or fewer forks and mor or fewer fins. Some fins may include one or more forking 22, while some may include none.

FIG. 24 demonstrates that the entire structure of the debris trap 200 can be manufactured separately from insulative cover on the shaft 2. The structure of the debris trap 200 can then be slid on and off the electrode through is the central cylindrical hole 23. The debris trap 200 may be coupled to the shaft 2 or insulative electrode cover by various means.

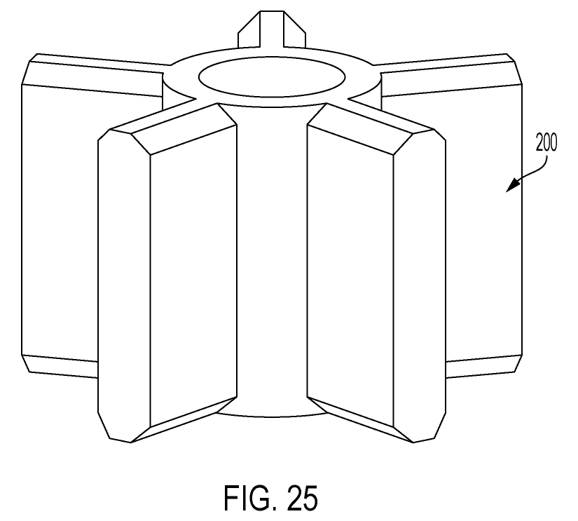

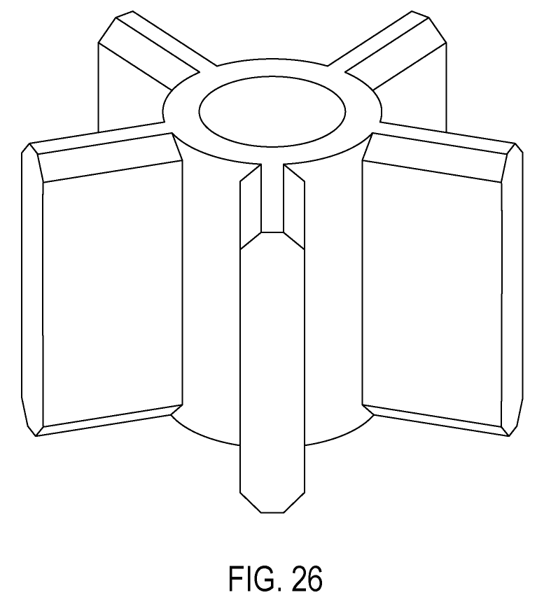

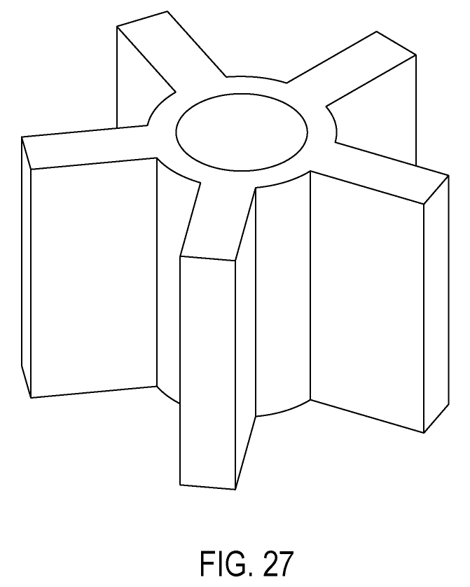

FIG. 25 depicts a CAD drawing of a debris trap 200 with fins that have chamfered cuts on upper and lower edge of fins. Specifically, the chamfered cuts may allow for a debris trap 200 with a lighter profile and/or one that is more easily removed.

FIG. 26 depicts a CAD drawing of a debris trap 200 with fins that have chamfered cuts on upper and lower edge of fins. Similar to the embodiment of FIG. 25, by having more severe chamfered cuts, additional benefits may be realized.

FIG. 27 depicts a CAD drawing of a debris trap 200 with fins that do not have chamfered cuts. As illustrated, the debris trap 200 may include a substantially circular center portion, with rectangular fins extending therefrom. Some embodiments may change the shape of the center portion (such as to rectangular, triangular, hexagonal, octagonal, etc.). Additionally, the fins may vary in shape, such as rounded.

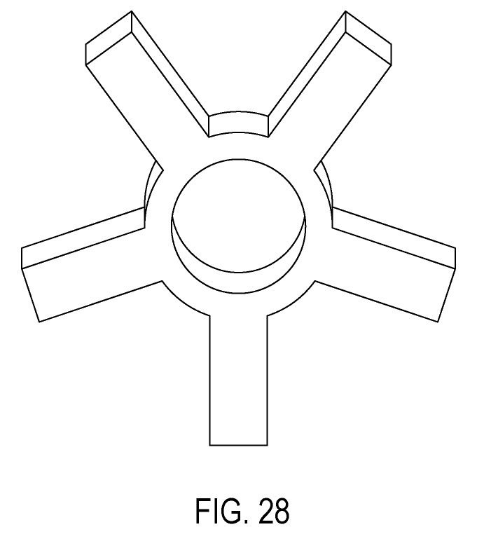

FIG. 28 depicts a CAD drawing of a debris trap 200 with fins that do not have chamfered cuts. Some embodiments include at least one aperture. In this embodiment, the central aperture is large, illustrating that the dimensions of the debris trap 200 components may vary, depending on the embodiment.

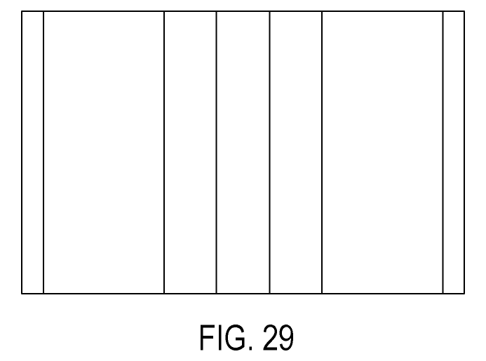

FIG. 29 depicts a side view of a debris trap 200 with fins that do not have chamfered cuts. As illustrated, some embodiments may be configured with a plurality of fins that extend from a central portion of the debris trap 200. In this embodiment, the fins may extent in a planar manner, and/or may be configured in a radial manner extending from the central portion.

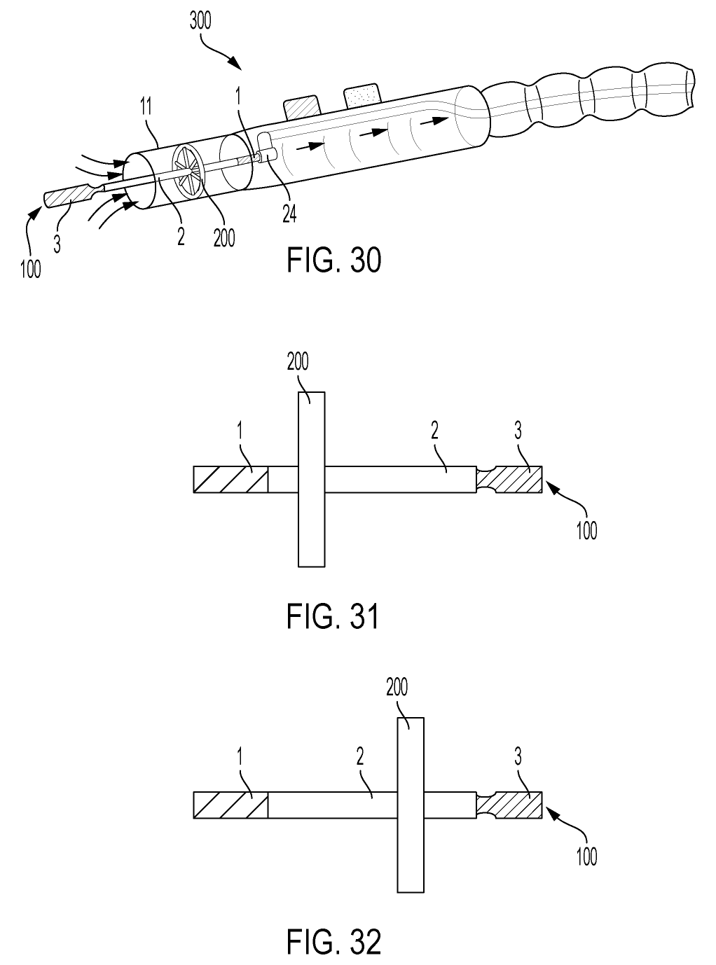

FIG. 30 depicts a smoke evacuation electrosurgical pencil 300 with smoke evacuation system. This cutaway drawing reveals the coupling mount 24 includes a socket into which the male connection end 1 is placed. The socket of the coupling mount 24 and male connection end 1 are both within the vacuum tube 11. The debris trap 200 may be located on the shaft 2 of the electrosurgical electrode blade 100 in the vacuum tube 11 between the male connection end 1 and the blade portion 3. The debris trap 200 may be positioned at the midpoint between male connection end 1 and blade portion 3 or it may be asymmetrically positioned between the two parts.

FIG. 31 depicts an electrosurgical electrode blade 100 with the debris trap 200 at an asymmetric point between the male connection end 1 and the blade portion 3. Some embodiments may have the debris trap 200 located at any on the electrode.

FIG. 32 depicts an electrosurgical electrode blade 100 with the debris trap 200 at an asymmetric point between the male connection end 1 and the blade portion 3. While the embodiment of FIG. 31 illustrates the debris trap 200 toward the male connection end 1, the embodiment of FIG. 32 depicts the debris trap 200 toward the blade portion 3.

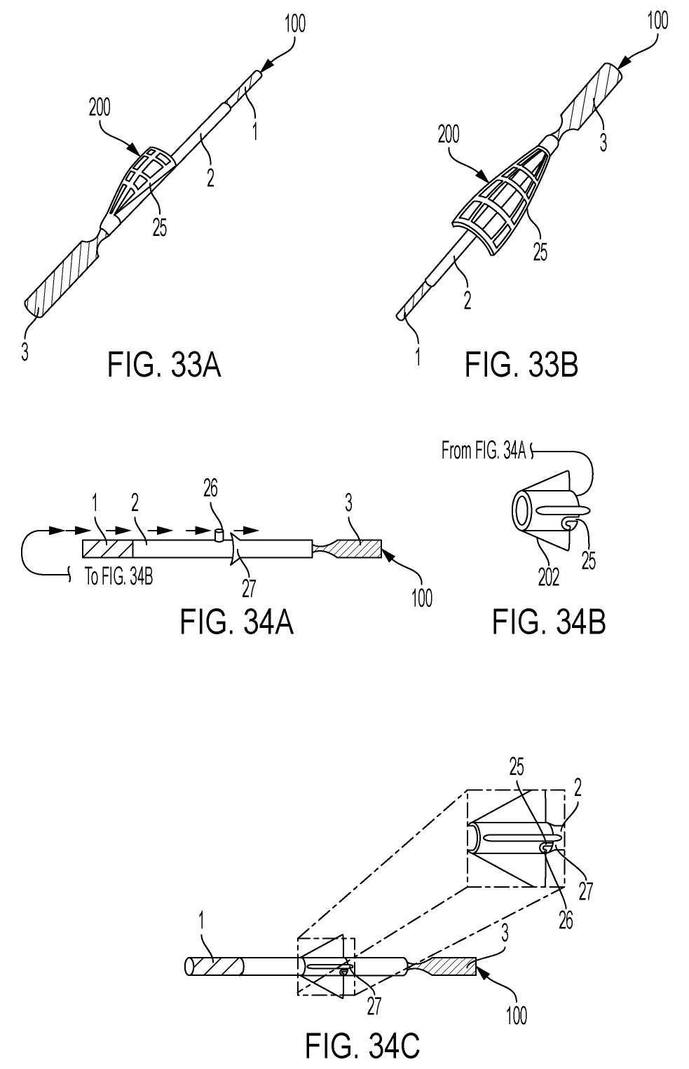

FIG. 33A depicts a side view of a curved net 25 that is connected to the electrode via an attachment mechanism. The curved net 25 may be part of a debris trap 200 and may radially cover a portion of the electrode. The curved net 25 is configured for smoke evacuation pencils having the electrosurgical electrode blade 100 asymmetrically positioned inside the vacuum tube 11.

FIG. 33B depicts another side view of the curved net 25 of FIG. 33A. As illustrated, the curved net 25 may be of a conical shape that extends around the circumference of the electrode. In some embodiments, the curved net 25 may only extend around a portion of the electrosurgical electrode blade 100. As will be understood, the debris trap 200 may be fixedly or removably coupled to the electrosurgical electrode blade 100.

FIG. 34A depicts an example locking mechanism for securing a debris trap 200 that is configured to removably couple to the electrosurgical electrode blade 100. In FIG. 34A, the electrosurgical electrode blade 100 may include a peg 26 on the shaft 2 and a widening portion 27 of the insulative cover for the shaft 2. Additional locking and coupling mechanism embodiments may include but are not limited to a snap-fit engagement, an injection molded press fit, threaded engagement, magnetic locking, tapered fit, cam-lock or lever engagement. The debris trap 200 may include a quick release button or a reusable snap latch.

For example, FIG. 34B depicts a separate debris trap 202 with a bayonet-style coupling groove 47, into which peg 26 from FIG. 34A can securely engage and lock. This allows a user to removably secure the debris trap 202 to the electrosurgical electrode blade 100. FIG. 34C depicts the separate debris trap 202 fully coupled and locked onto peg 26.

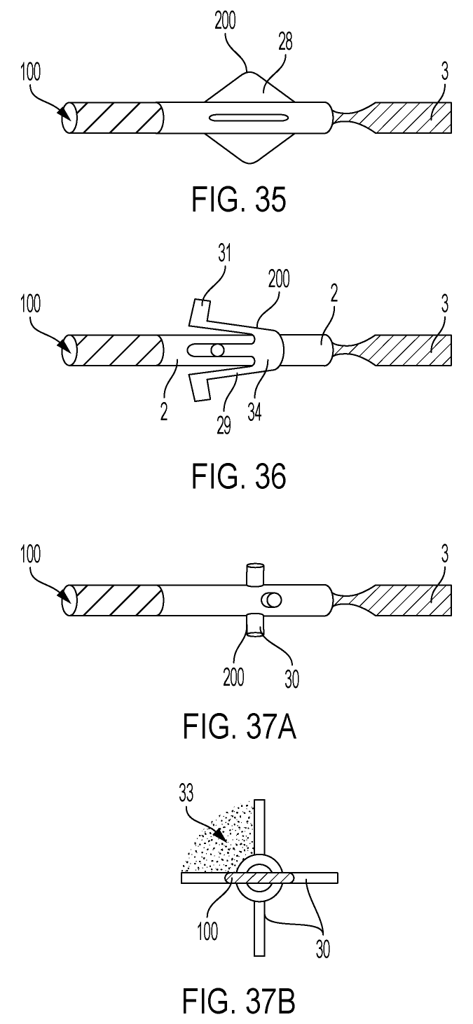

FIG. 35 depicts another embodiment of a debris trap 200. As illustrated, the debris trap 200 may include fins 28 coupled to the shaft 2. Each of the fins 28 may individually have a shape selected from, for example, triangle, rectangular, trapezoidal, elliptical, clipped delta, or wedge- shaped.

FIG. 36 depicts another embodiment of a debris trap 200 with prongs 29 and a securing portion 34 that engages with the shaft 2. The prongs 29 may be configured to extend outward from the shaft 2 at an oblique angle, directed away from the blade portion 3. The prongs 29 include an angled distal portion 31 that is directed away from the shaft 2 and directed toward the vacuum tube 11. The geometry of the prongs 29 allows close conformity with smoke evacuation pencils having a conical geometry at the working end, where the electrode coupling is located. Additional embodiments may include but are not limited to flexible prongs or curved prongs.

FIG. 37A and 37B depict a debris trap 200 that includes fins 30 and vanes 33 existing between the fins 30. The fins 30 may be cylindrical or other shape and may provide an aspect ratio from 20:1 to 2:1, such as from 10:1 to 2:1, from 5:1 to 2:1, from 3.75:1 to 2:1, from 20:1 to 5:1, from 20:1 to 10:1, or from 10:1 to 5:1. The aspect ratio is determined by dividing the fin's span (height) by its average width. The vanes 33 are highlighted by the shaded area.

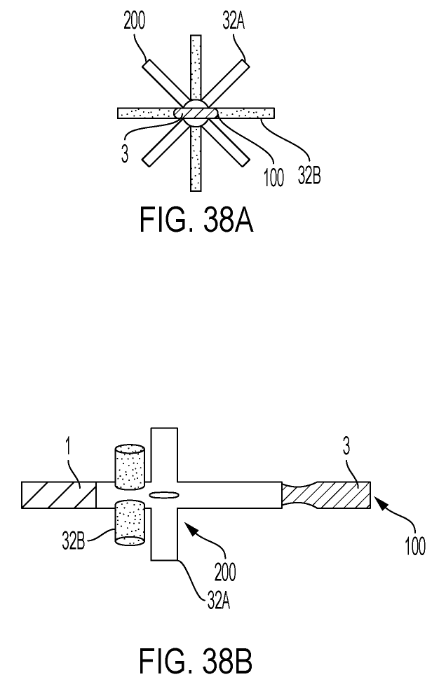

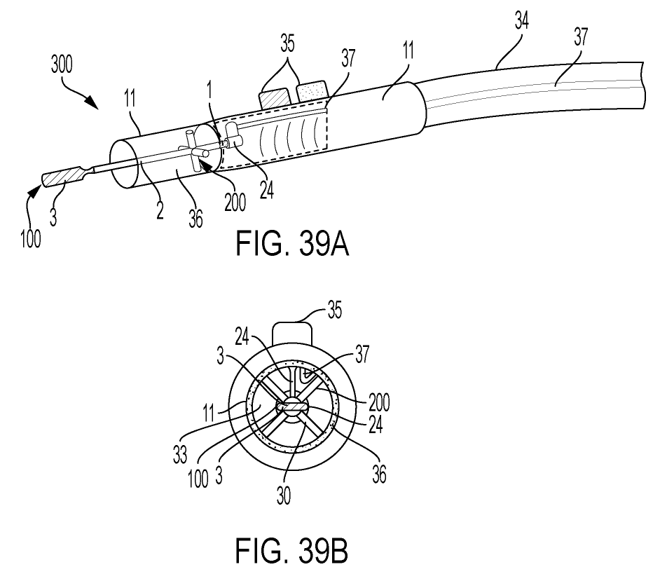

FIGS. 38A and 38B depict a debris trap 200 comprising multiple rows of fins. The first row of fins 32A may be positioned toward the blade portion 3, while the additional row of fins 32B may be positioned between the debris trap 200 and the male connection end 1 of the electrode. The additional row of fins 32B is illustrated using darker shading for clarity. Some embodiments may include any number of fins and rows of fins with variable numbers of fins.

As disclosed herein and in embodiments, the debris trap 200 may be positioned within the vacuum tube 11. Debris trap 200 may incorporate various structural designs and feature either open or closed architecture. It is contemplated that the debris trap 200 may be fixedly or removably coupled to the vacuum tube 11. For example, the debris trap 200 may be positioned within vacuum tube 11, circumferencing the electrode without impairing visualization of the electrode tip or critical anatomical structures within the surgical field. Additionally, the debris trap 200 may also be constructed from translucent insulative material, further reducing visual obstruction. A vacuum tube 11 that includes a debris trap 200 is suitable for a smoke evacuation electrosurgical pencil 300 in which the vacuum tube 11 is removable and/or disposable.

FIG. 39A depicts a smoke evacuation electrosurgical pencil 300 with a cutout window 310 outlined by dotted lines, illustrating the vacuum tube 11 interior. The electrosurgical electrode blade 100 is coupled to the smoke evacuation electrosurgical pencil 300 by a coupling mount 24 which extends from the wall of the vacuum tube 11. Activation buttons 35 are disposed on an outer surface. Power source wire 37 electrically contacts the male connection end 1 within coupling mount 24. The distal end 36 of vacuum tube 11 is translucent. In this depiction the distal end 36 is illustrated with darker shading for clarity. Electrosurgical electrode blade 100 includes a debris trap 200 attached to its shaft 2. Debris trap 200 is positioned closer to the male connection end 1 of the electrosurgical electrode blade 100 to prevent visual obstruction of blade portion 3. FIG. 39B provides an axial view of this smoke evacuation electrosurgical pencil 300 clearly showing the vanes 33, which may be low aspect ratio air vanes created by the debris trap 200. The debris trap 200 is located on electrosurgical electrode blade 100 and within vacuum tube 11. The power source wire 37 is connected to a radiofrequency generator.

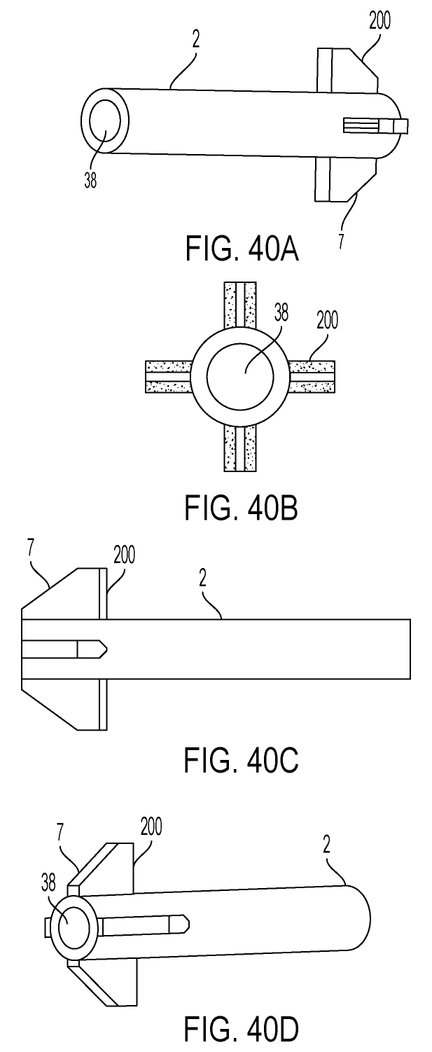

FIGS. 40A, 40B, 40C and 40D depict a plurality of views of an insulative electrode cover comprising a debris trap 200 without an electrode present. The insulative cover may be formed from translucent materials to reduce visual obstruction. The internal lumen 38 of the insulative cover is dimensioned to accommodate an electrosurgical electrode blade 100.

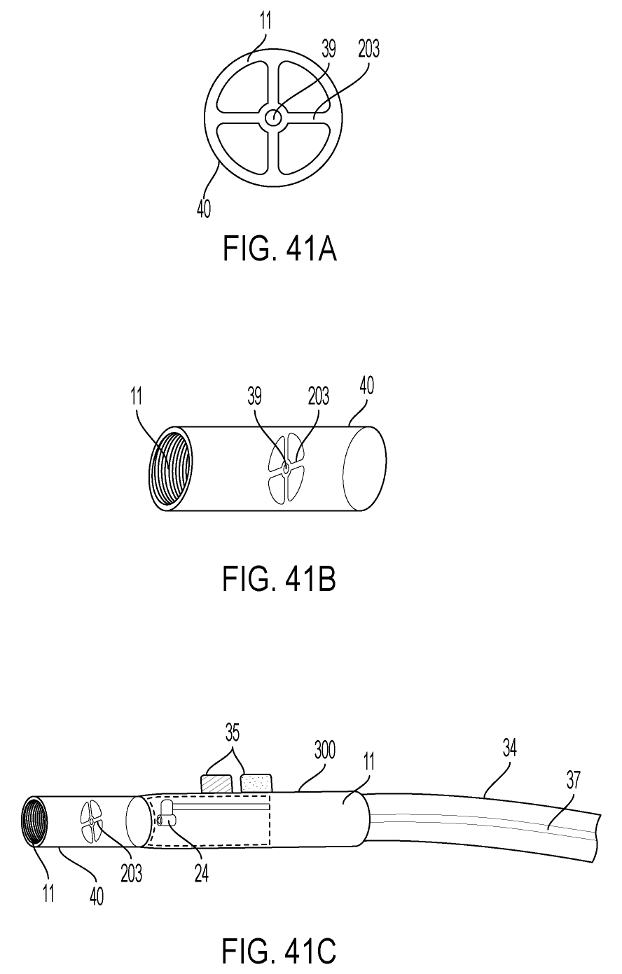

FIG. 41A depicts an axial view of a removable vacuum tube 40 incorporating a debris trap 203 without an electrode. The debris trap 203 may include an internal lumen 39 which can accommodate an electrode. The debris trap 200 may utilize one of various designs situated at any location within or outside of the removable vacuum tube. FIG. 41B depicts an oblique view of the debris trap 203 of FIG. 41A without an electrode. The internal lumen 39 is dimensioned to accommodate an electrosurgical electrode blade 100. FIG. 41C depicts a smoke evacuation electrosurgical pencil 300 fitted with a removable vacuum tube containing a debris trap 203, depicted without an electrode. The removable vacuum tube 40 and debris trap 203 may be formed from translucent materials to reduce visual obstruction.

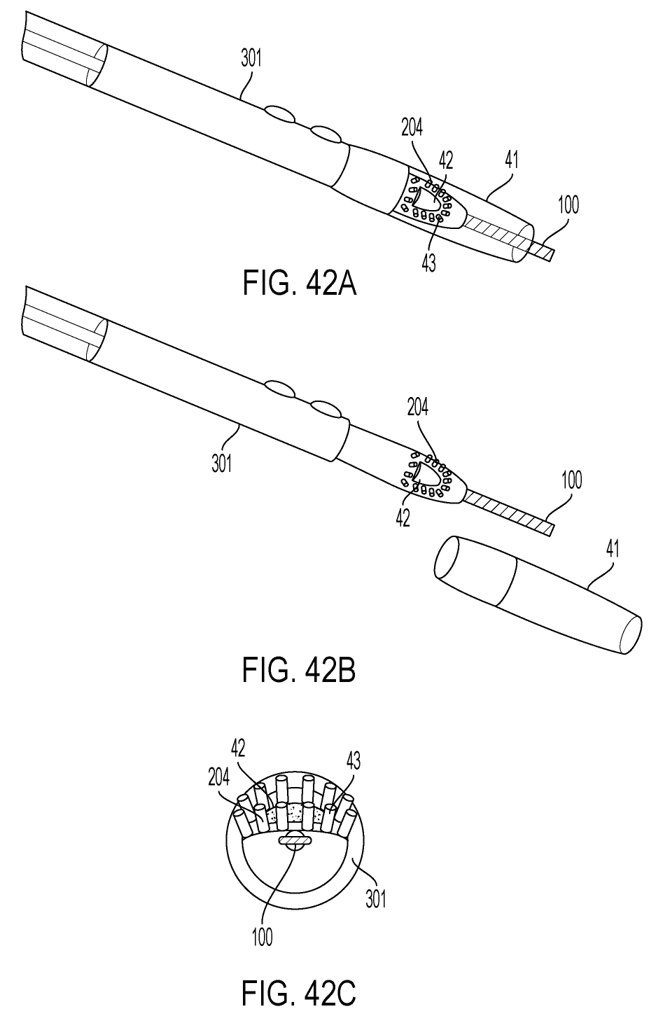

FIG. 42A depicts a smoke evacuation electrosurgical pencil 301 featuring a debris trap 204 with a built-in configuration positioned around the opening of the vacuum tube lumen 42. Debris trap 204 may include a plurality of outward extending pegs 43. A removable vacuum cap 41 may be attached to the smoke evacuation electrosurgical pencil 301. When the removable vacuum cap 41 is engaged with the smoke evacuation electrosurgical pencil 301, pegs 43 may align flush with the inner wall of the removable vacuum cap 41. FIG. 42B depicts the removable vacuum cap 41 detached from the smoke evacuation electrosurgical pencil 301.

FIG. 42C depicts an axial view of the smoke evacuation electrosurgical pencil 301 with built in debris trap 204 highlighting the vacuum tube lumen 42 as indicated by the shaded area. Another embodiment of the debris trap 204 may include a thin, flat structure positioned over the opening of the vacuum tube lumen 42. This debris trap 200 is part of the electrosurgical pencil and includes a pattern of openings or channels that allow air and surgical smoke to pass through while blocking larger debris. The channels are sized and arranged to maintain airflow efficiency but prevent the entry of debris that could obstruct the lumen.

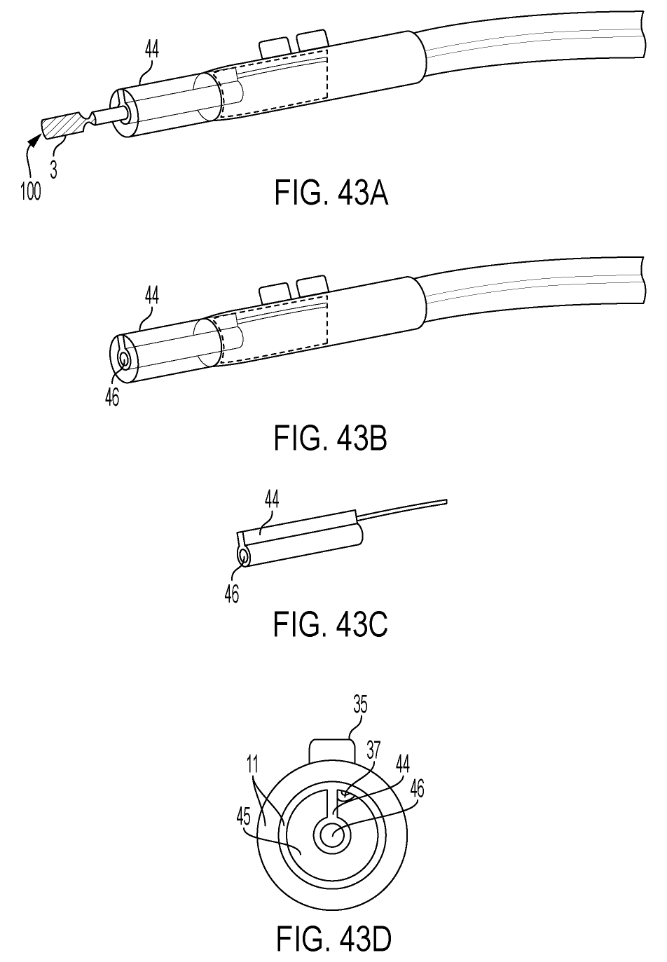

FIGS. 43A through 43D depict an embodiment of an electrosurgical pencil incorporating a smoke evacuation system with an integrated electrode connector sheath. As shown in FIG. 43A, the electrosurgical pencil includes a sheath 44 that is configured to attach to the internal wall of the vacuum tube 11. The sheath 44 extends from a proximal region where the electrosurgical power source connects to the electrosurgical electrode blade 100 and continues distally to the terminal end of the vacuum tube 11. The sheath 44 defines an internal lumen 46 configured to receive and house the electrosurgical electrode blade 100.

FIG. 43B depicts the same embodiment as in FIG. 43A, with the electrosurgical electrode blade 100 removed to better show the internal structure of the sheath 44 and the internal lumen 46. FIG. 43C depicts the sheath 44 and its connection to the power source with the remainder of the electrosurgical pencil assembly removed, illustrating how the sheath interfaces with the power delivery system independently of the pencil body. FIG. 43D provides an axial view of the embodiment, further detailing the spatial relationship between the sheath 44, the internal lumen 46, and the surrounding wall of the vacuum tube 11. The sheath 44 is comprised of an insulative material and can be translucent. Some embodiments may include a thin wall that extends radially from the internal wall of the vacuum tube 11 toward the electrode. This wall has a diameter approximately equal to that of the electrode and is positioned such that the electrode shaft rests closely against or within it.

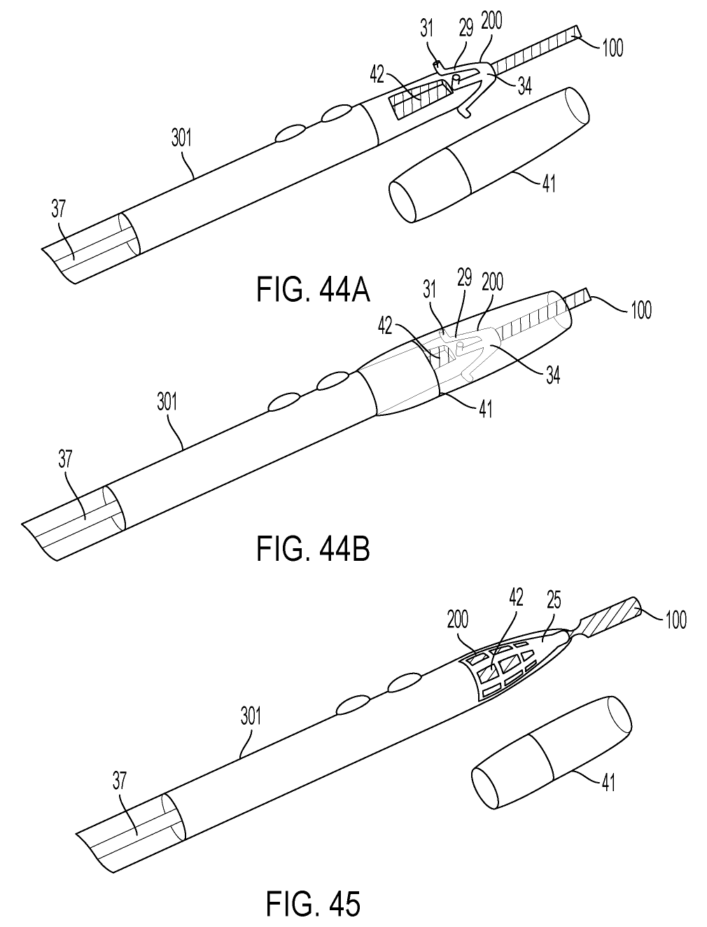

FIG. 44A depicts an smoke evacuation electrosurgical pencil 301 featuring a vacuum tube lumen 42 that is non-concentric. An electrosurgical electrode blade 100 is shown with a debris trap 200 that is attached and includes prongs 29 that conform to the distal end of the smoke evacuation electrosurgical pencil 301. A removable vacuum cap 41 is depicted in a detached configuration from the smoke evacuation electrosurgical pencil 301. FIG. 44B shows the removable vacuum cap 41 coupled with the smoke evacuation electrosurgical pencil 301. When the removable vacuum cap 41 is secured, the angled distal portion 31 of the prongs 29 closely conform to the inner surface of the removable vacuum cap 41, forming a physical barrier that acts as a filter to capture larger debris that may be incidentally suctioned, thereby preventing such debris from entering the vacuum tube lumen 42. The number of prongs and prong size may vary, and the spacing between prongs may be consistent or variable to accommodate different geometries or functional requirements.

FIG. 45 depicts an smoke evacuation electrosurgical pencil 301 featuring a debris trap 200 with net-like properties positioned over the vacuum tube lumen 42 and coupled to an electrosurgical electrode blade 100. A removable vacuum cap 41 is shown in a detached configuration from the smoke evacuation electrosurgical pencil 301. In this embodiment, the debris trap 200 is spaced proximally from the electrode tip, thereby preserving an unobstructed view of the electrosurgical electrode blade 100 during use and minimizing interference with surgical visualization.

From the above, it is to be appreciated that defined herein is an electrode for an electrosurgical pencil. The electrode includes a debris trap 200 coupled to a shaft of the electrode. The debris trap includes a plurality of trapping structures. The aspects of the electrode described herein offer several advantages over other electrodes. Incorporating a debris trap directly into the central shaft of the electrode blade allows the debris trap to capture and contain larger debris before it enters the electrosurgical pencil assembly, preventing blockages that could otherwise disrupt the surgical smoke evacuation system. The design of the electrode blade allows it to be easily removed, replaced, and interchanged for different surgical procedures, providing the surgical team with flexibility and adaptability in their toolset.

Further aspects of the aspects described herein are provided by the subject matter of the following clauses:

A first aspect includes smoke evacuation electrosurgical pencil comprising: a vacuum tube that is substantially cylindrical and includes an inner surface, the vacuum tube configured for vacuuming debris during use of the smoke evacuation electrosurgical pencil in a surgical procedure; an electrosurgical electrode blade that is partially disposed within the vacuum tube and partially extending from an end of the vacuum tube, the electrosurgical electrode blade configured for performing the surgical procedure, the electrosurgical electrode blade including a shaft that is disposed in the vacuum tube; and a debris trap coupled to the shaft and removably disposed within the vacuum tube, the debris trap extending to the inner surface of the vacuum tube and including a plurality of trapping structures to receive and capture debris resulting from the surgical procedure.

A second aspect includes the first aspect, wherein the debris trap includes a plurality of fins extending from a center portion of the debris trap.

A third aspect includes the first aspect and/or the second aspect, wherein the debris trap includes a ring that surrounds a center portion of the debris trap.

A fourth aspect includes any of the first aspect through the third aspect, wherein the debris trap includes at least one tapered fin extending from a center portion of the debris trap.

A fifth aspect includes any of the first aspect through the fourth aspect, wherein the debris trap is configured in a disk-like configuration.

A sixth aspect includes any of the first aspect through the fifth aspect, wherein the debris trap includes at least one aperture.

A seventh aspect includes any of the first aspect through the sixth aspect, wherein the debris trap is configured as a cup-like configuration.

An eighth aspect includes any of the first aspect through the seventh aspect, wherein the debris trap includes a curved fin.

A ninth aspect includes any of the first aspect through the eighth aspect, wherein the debris trap includes at least one notched cutout.

A tenth aspect includes any of the first aspect through the ninth aspect, wherein the debris trap includes at least one prong.

An eleventh aspect includes any of the first aspect through the tenth aspect, wherein the debris trap includes at least one forking.

A twelfth aspect includes any of the first aspect through the eleventh aspect, wherein the debris trap is configured with a curved net.

A thirteenth aspect includes any of the first aspect through the twelfth aspect, wherein the debris trap includes a coupling groove for engaging with a notch on the shaft.

A fourteenth aspect includes any of the first aspect through the thirteenth aspect, wherein the debris trap is coated with at least one of the following: a non-stick or a hydrophobic material.

A fifteenth aspect includes a smoke evacuation electrosurgical pencil comprising: a vacuum tube that is substantially cylindrical and includes an inner surface, the vacuum tube configured for vacuuming debris during use of the smoke evacuation electrosurgical pencil in a surgical procedure; an electrosurgical electrode blade that is partially disposed within the vacuum tube and partially extending from an end of the vacuum tube, the electrosurgical electrode blade coupled to the smoke evacuation electrosurgical pencil by a coupling mount that extends from a wall of the vacuum tube, the electrosurgical electrode blade configured for performing the surgical procedure, the electrosurgical electrode blade including a shaft that is disposed in the vacuum tube; and a debris trap coupled to the shaft and removably disposed within the vacuum tube, the debris trap extending to the inner surface of the vacuum tube and including a plurality of trapping structures to receive and capture debris resulting from the surgical procedure.

A sixteenth aspect includes the fifteenth aspect, wherein the debris trap includes at least one of the following: a plurality of fins extending from a center portion of the debris trap, a ring that surrounds the center portion of the debris trap, at least one tapered fin extending from the center portion of the debris trap, at least one aperture, a curved fin, at least one notched cutout, at least one prong, at least one forking, or net configuration.

A seventeenth aspect includes the fifteenth aspect and/or the sixteenth aspect, wherein the debris trap includes a coupling groove for engaging with a notch on the shaft.

An eighteenth aspect includes any of the fifteenth aspect through the seventeenth aspect, wherein the debris trap is configured as at least one of the following: a cup-like configuration or a disk-like configuration.

A nineteenth aspect includes a smoke evacuation electrosurgical pencil comprising: a vacuum tube that is substantially cylindrical and includes an inner surface, the vacuum tube configured for vacuuming debris during use of the smoke evacuation electrosurgical pencil in a surgical procedure; an electrosurgical electrode blade that is partially disposed within the vacuum tube and partially extending from an end of the vacuum tube, the electrosurgical electrode blade coupled to the smoke evacuation electrosurgical pencil by a coupling mount that extends from a wall of the vacuum tube, the electrosurgical electrode blade configured for performing the surgical procedure, the electrosurgical electrode blade including a shaft that is disposed in the vacuum tube; and a debris trap coupled to the shaft and removably disposed within the vacuum tube, the debris trap extending to the inner surface of the vacuum tube and including a plurality of trapping structures to receive and capture debris resulting from the surgical procedure, wherein the debris trap includes a coupling groove for engaging with a notch on the shaft.

A twentieth aspect includes the nineteenth aspect, wherein the debris trap includes at least one of the following: a plurality of fins extending from a center portion of the debris trap, a ring that surrounds the center portion of the debris trap, at least one tapered fin extending from the center portion of the debris trap, at least one aperture, a curved fin, at least one notched cutout, at least one prong, at least one forking, or net configuration.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A smoke evacuation electrosurgical pencil comprising:
a vacuum tube that is substantially cylindrical and includes an inner surface, the vacuum tube configured for vacuuming debris during use of the smoke evacuation electrosurgical pencil in a surgical procedure;
an electrosurgical electrode blade that is partially disposed within the vacuum tube and partially extending from an end of the vacuum tube, the electrosurgical electrode blade configured for performing the surgical procedure, the electrosurgical electrode blade including a shaft that is disposed in the vacuum tube; and
a debris trap coupled to the shaft and removably disposed within the vacuum tube, the debris trap extending to the inner surface of the vacuum tube and including a plurality of trapping structures to receive and capture debris resulting from the surgical procedure.

2. The smoke evacuation electrosurgical pencil of claim 1, wherein the debris trap includes a plurality of fins extending from a center portion of the debris trap.

3. The smoke evacuation electrosurgical pencil of claim 1, wherein the debris trap includes a ring that surrounds a center portion of the debris trap.

4. The smoke evacuation electrosurgical pencil of claim 1, wherein the debris trap includes at least one tapered fin extending from a center portion of the debris trap.

5. The smoke evacuation electrosurgical pencil of claim 1, wherein the debris trap is configured in a disk-like configuration.

6. The smoke evacuation electrosurgical pencil of claim 1, wherein the debris trap includes at least one aperture.

7. The smoke evacuation electrosurgical pencil of claim 1, wherein the debris trap is configured as a cup-like configuration.

8. The smoke evacuation electrosurgical pencil of claim 1, wherein the debris trap includes a curved fin.

9. The smoke evacuation electrosurgical pencil of claim 1, wherein the debris trap includes at least one notched cutout.

10. The smoke evacuation electrosurgical pencil of claim 1, wherein the debris trap includes at least one prong.

11. The smoke evacuation electrosurgical pencil of claim 1, wherein the debris trap includes at least one forking.

12. The smoke evacuation electrosurgical pencil of claim 1, wherein the debris trap is configured with a curved net.

13. The smoke evacuation electrosurgical pencil of claim 1, wherein the debris trap includes a coupling groove for engaging with a notch on the shaft.

14. The smoke evacuation electrosurgical pencil of claim 1, wherein the debris trap is coated with at least one of the following: a non-stick or a hydrophobic material.

15. A smoke evacuation electrosurgical pencil comprising:
a vacuum tube that is substantially cylindrical and includes an inner surface, the vacuum tube configured for vacuuming debris during use of the smoke evacuation electrosurgical pencil in a surgical procedure;
an electrosurgical electrode blade that is partially disposed within the vacuum tube and partially extending from an end of the vacuum tube, the electrosurgical electrode blade coupled to the smoke evacuation electrosurgical pencil by a coupling mount that extends from a wall of the vacuum tube, the electrosurgical electrode blade configured for performing the surgical procedure, the electrosurgical electrode blade including a shaft that is disposed in the vacuum tube; and
a debris trap coupled to the shaft and removably disposed within the vacuum tube, the debris trap extending to the inner surface of the vacuum tube and including a plurality of trapping structures to receive and capture debris resulting from the surgical procedure.

16. The smoke evacuation electrosurgical pencil of claim 15, wherein the debris trap includes at least one of the following: a plurality of fins extending from a center portion of the debris trap, a ring that surrounds the center portion of the debris trap, at least one tapered fin extending from the center portion of the debris trap, at least one aperture, a curved fin, at least one notched cutout, at least one prong, at least one forking, or net configuration.

17. The smoke evacuation electrosurgical pencil of claim 15, wherein the debris trap includes a coupling groove for engaging with a notch on the shaft.

18. The smoke evacuation electrosurgical pencil of claim 15, wherein the debris trap is configured as at least one of the following: a cup-like configuration or a disk-like configuration.

19. A smoke evacuation electrosurgical pencil comprising:
a vacuum tube that is substantially cylindrical and includes an inner surface, the vacuum tube configured for vacuuming debris during use of the smoke evacuation electrosurgical pencil in a surgical procedure;
an electrosurgical electrode blade that is partially disposed within the vacuum tube and partially extending from an end of the vacuum tube, the electrosurgical electrode blade coupled to the smoke evacuation electrosurgical pencil by a coupling mount that extends from a wall of the vacuum tube, the electrosurgical electrode blade configured for performing the surgical procedure, the electrosurgical electrode blade including a shaft that is disposed in the vacuum tube; and
a debris trap coupled to the shaft and removably disposed within the vacuum tube, the debris trap extending to the inner surface of the vacuum tube and including a plurality of trapping structures to receive and capture debris resulting from the surgical procedure, wherein the debris trap includes a coupling groove for engaging with a notch on the shaft.

20. The smoke evacuation electrosurgical pencil of claim 19, wherein the debris trap includes at least one of the following: a plurality of fins extending from a center portion of the debris trap, a ring that surrounds the center portion of the debris trap, at least one tapered fin extending from the center portion of the debris trap, at least one aperture, a curved fin, at least one notched cutout, at least one prong, at least one forking, or net configuration.
